(19) United States
(12) Patent Application Publication
(10) Pub. No.: US 2021/0386041 A1
(43) Pub. Date: Dec. 16, 2021

(54) MITE COMPOSITION AND METHOD FOR REARING MITES

(71) Applicant: BIOBEST GROUP N.V., Westerlo (BE)

(72) Inventors: Dominiek VANGANSBEKE, Westerlo (BE); Marcus Vinicius Alfenas DUARTE, Westerlo (BE); Manon Hélène Lionella GUILBAUD, Westerlo (BE); Alfredo BENAVENTE MARTINEZ, Westerlo (BE); Apostolos PEKAS, Westerlo (BE); Karel Jozef Florent BOLCKMANS, Westerlo (BE); Felix Leopold WÄCKERS, Westerlo (BE)

(21) Appl. No.: 17/281,806

(22) PCT Filed: Oct. 7, 2019
(86) PCT No.: PCT/EP2019/077115

§ 371 Date: Mar. 31, 2021

(30) Foreign Application Priority Data
Oct. 5, 2018 (BE) BE2018/5688

(51) Int. Cl. A01K 67/033

(52) U.S. Cl. CPC A01K 67/033; A01K 2227/706

(57) ABSTRACT

The present invention relates to methods for the mass rearing of predatory mites, mite compositions and the use thereof for biological control. In particular, the present invention relates to the use of tarsonemid prey mites for the mass rearing of predatory mites and for controlling a pest in a crop.

### FIELD OF THE INVENTION

The present invention relates to methods for the mass rearing of predatory mites, mite compositions and the use thereof for biological control. In particular, the present invention relates to the use of tarsonemid prey mites for the mass rearing of predatory mites and for controlling a pest in a crop.

### BACKGROUND OF THE INVENTION

Predatory mites are traditionally used in the broad field of agricultural pest management. A wide range of predatory mite species have been suggested or commercialized for the biological control of phytophagous pest mites and insect pests such as whiteflies and thrips.

In recent years, the major arthropod pest species involved have generally been controlled by chemical agents i.e. pesticides. Many newer pesticides made available in the past decade are more selective and less hazardous than most of the older compounds. However, it is becoming increasingly clear that the strategy of unilateral reliance on chemical control will not be the solution to the problem. In this regard, there are several major problems attendant to chemical control i.e. the development of resistance to chemicals in target pest species; the dwindling supply of useful, registered insecticides and acaricides; the deposit of some undesirable residues; the damaging (or detrimental) effect of these chemicals on non-target species resulting in secondary pest outbreaks (contaminate soil and water affect wildlife, aquatic life, and other non-target organisms and interfere with beneficial organisms such as pollinating insects and the natural enemies of pests); and the phytotoxic reactions by treated plants. For these reasons, many farmers and gardeners are exploring and adopting methods that reduce pesticide use.

Biological control represents one alternative to the use of chemical agents. Biological control is the intentional manipulation of populations of living beneficial organisms (natural enemies) in order to limit population of pests. Virtually all pests have natural enemies and appropriate management of natural enemies can effectively control many pests. Biological control can be effective, economical and safe. The intent of biological control is not to eradicate pests, but to keep them at tolerable levels at which they cause no appreciable damage.

Natural enemies of mites include predators, parasitic insects, nematodes, and pathogens. Many publications already demonstrate the use of a predatory mite population in order to control pests. For example, currently, primarily phytoseiid predatory mites are employed to combat pests such as phytophagous mites, thrips and whiteflies. As one example, Neoseiulus cucumeris (Oudemans) is commercially used for the control of thrips larvae and spider mites. In addition, other predatory mite species selected from mesostigmatid and prostigmatid predatory species receive attention in biological pest control and some have entered market.

It is therefore an interest of developing effective mass rearing systems to produce predatory mite populations on a commercially relevant scale for an acceptable price. In the present commercial rearing systems populations of the predatory mites are reared on life prey in a culture maintained on a carrier. For example, WO2006071107 and WO2013103295 disclose a mite composition comprising a population of individuals of a predatory mite species, a prey mite population as a food source for the predatory mite individuals and a carrier. The composition according to this prior art document is suitable for rearing a mite species and for the biological control of pests. The most commonly used prey mites in the mass rearing methods of the prior art are Astigmatid mites, such as Tyrophagus putrescentiae (Schrank), Thyreophagus entomophagous (Laboulbéne & Robin) and Carpoglyphus lactis L. Up to date, all prey mite species being used in commercial mass-rearing of predatory mites belong to the order Astigmata. For example, WO 2006/056552, WO 2008/015393, WO 2008/104807 and WO 2007/075081 demonstrate the potential of Astigmatids to be used as prey mites in mass-rearing of predatory mites.

Pest mites are sometimes used to study the interaction between predatory mites and the pest mites. For example, Polyphagotarsonemus latus (Rodriguez Morell et al., 2010, International Journal of Acarology 36 (5): 371-376) and Phytonemus pallidus (Tuovinen & Lindqvist, 2010, Biological Control 54 (2): 119-125) mites from the family of Tarsonemidae were grown on lab-scale on living plant material and were used to study attacks by predatory mites. However, mite compositions with such prey mite populations are not a realistic solution for commercial culturing since these are known as pests that should not be distributed.

Mass rearing systems for predatory mites depend heavily on the availability of suitable prey for the predators. In view of their role in rearing of predatory mites, the commercial relevance of rearing prey mites is increasing.

In view of the above there is a continuing need to obtain improved (more efficient) mite compositions for mass rearing and large-scale production of predatory mite populations, for commercial distribution of larger volumes of mite compositions comprising such and for better pest control and agricultural management.

In addition, commercial mite rearing requires rearing conditions at a substantial temperature (generally between 20 and 30° C.) and high humidity (generally between 80 and 95% relative humidity). In these conditions, problems of fungal contamination easily occur. Therefore, there is also need for improved mite rearing methods that lower the risk of fungal contamination.

### SUMMARY OF THE INVENTION

The inventors have surprisingly found that the use of tarsonemids as prey mites overcome the problems of the prior art.

Therefore the present invention provides a mite composition comprising
a predatory mite population,
a prey mite population, and
a carrier for individuals of said populations,
   wherein said prey mite population comprises mites from the family of the Tarsonemidae, provided that said mite composition does not comprise a living mite species selected from the group consisting of Polyphagotarsonemus latus and Phytonemus pallidus.

### BRIEF DESCRIPTION OF THE DRAWINGS

FIG. 1: Number of eggs deposited by a female A. swirskii for 3 consecutive days when fed T. fusarii (TF), C. lactis (CL) or T. entomophagus (TE) at 22±1° C. and 85±5% RH. Different letters above the bars denote significant differences among treatments (contrasts after GLM, p<0.05).

FIG. 2: Capture success rate of A. swirskii females on eggs, larvae and adults of T. fusarii (TF), C. lactis (CL) and T. entomophagus (TE). Different letters indicate significant differences between the prey mite species per developmental stage (contrasts after GLM, p<0.05).

FIG. 3: Number of eggs deposited by female H. anconai for 3 consecutive days when fed T. fusarii as compared to a control consisting only of a tomato leaf arena (22±1° C. and 85±5% RH).

FIG. 4: Number of eggs deposited by female P. ubiquitus for 3 consecutive days when fed T. fusarii as compared to C. lactis and a control treatment without prey mites (22±1° C. and 85±5% RH).

FIG. 5: Number of eggs deposited by female A. swirskii for 2 consecutive days when fed either T. fusarii or T. confusus (22±1° C. and 85±5% RH).

FIG. 6: Number of eggs deposited by female A. swirskii for 2 consecutive days when fed Fungitarsonemus sp. as compared to a control consisting only of an ivy leaf arena (22±1° C. and 85±5% RH).

### DETAILED DESCRIPTION OF THE INVENTION

As described herein before, the present invention provides a mite composition comprising
a predatory mite population,
a prey mite population, and
a carrier for individuals of said populations,
   wherein said prey mite population comprises mites from the family of the Tarsonemidae. In particular such a composition wherein said mite composition does not comprise a living mite species selected from the group consisting of Polyphagotarsonemus latus and Phytonemus pallidus.

By the term «predatory mite population», is meant in the sense of the present invention, a population of beneficial mites that feeds on a prey mite population.

By the term «prey mite population», is meant in the sense of the present invention, a population of mites that at least partially consumed by a predatory mite population.

By the term «carrier», is meant in the sense of the present invention, any solid material which is suitable to provide a carrier surface to the individuals of both the predatory and the prey mite populations. The carrier will usually act as a three-dimensional matrix wherein the prey mite population and predatory mite population can move around, hide, develop and prey or be preyed upon.

Inventors surprisingly observed that a mite composition according to the invention shows better results in terms of efficiency. It was observed that the mites from the family of Tarsonemidae of the prey mite population are more easily killed and more readily eaten by the predatory mite population than the conventionally used astigmatid prey mites. An additional advantage is that all life stages of the tarsonemid prey mites are being killed readily, whereas larger astigmatid life stages, such as adults, are being killed at a much lower rate. In addition, it was observed that at even at high densities, the prey mite population disturbs the predatory mite population less than the astigmatid prey mites used for mass rearing mites. Although not wishing to be bound by theory, it appears that the tarsonemid mites might not produce repellent volatiles or produce them to a much lower degree, compared to astigmatid mites. This may cause the tarsonemids to have a lower disturbing effect on the predatory mite population. Accordingly, individuals from the predatory mite population may grow faster and therefore mature more quickly which allows for a higher rate of reproduction. Such a higher reproductive rate results in a faster increase in the number of individuals in the predatory mite population. This advantage is beneficial for the rearing of predatory mites and the production of compositions comprising such, as well as for the control of a pest in a crop. It is to be noted that the prey mite population used in the invention may also comprise (or consist of) a population of dead prey mites. The observation that the prey mites of the invention are more readily eaten than the prey mites of the prior art, such as astigmatid mites, also applies to dead tarsonemid mites in comparison to dead astigmatid mites. The use of a prey mite population comprising dead prey mites provides the benefit that predatory mites require less energy for killing and consuming the prey mites.

Secondly, it was observed that the appearance of mold is prevented with the mite composition according to the invention. Indeed, it was observed that mites from the family of Tarsonemidae feed on fungi. Surprisingly, the tarsonemid prey mites not only reduce fungal growth, but are able to completely suppress it to a non-visible degree in commercially relevant rearing conditions. Indeed, as discussed herein before, for the rearing of predatory mites for the purpose of production and distribution, it is essential to maintain strict conditions of temperature and humidity. A hot and humid environment is ideal for the rearing of predatory mites. Unfortunately, such specific conditions favor fungal growth, which severely limit the ability to rear mites and to produce commercially acceptable predatory mite compositions. The composition according to the invention, contrary to mite compositions from the state of the art, allows mass rearing and thus commercial distribution of larger volumes, both leading to cost reduction. Therefore, preferably, the prey mite population comprises living mites from the family of the Tarsonemidae. The use of living mites according to the invention provides a number of benefits over the use of dead mites, such as the suppression of fungal contamination.

In a particular embodiment, the prey mite population as used in the invention comprises (a) a population of dead prey mites, and (b) a population of living mites from the family of the Tarsonemidae. As the use of dead prey mites often cause fungal contamination of rearing cultures, the addition of living prey mites according to the invention suppresses this problem. In a more particular embodiment, the prey mite population as used in the invention comprises (a) a population of dead prey mites from the family of the Tarsonemidae, and (b) a population of living mites from the family of the Tarsonemidae.

By the term «mold», is meant in the sense of the present invention, a fungus that grows in the form of multicellular filaments called hyphae. Molds are a large and taxonomically diverse number of fungal species causing biodegradation of natural materials. Molds do not form a specific taxonomic or phylogenetic grouping but can be found in the divisions Zygomycota and Ascomycota. Common genera of molds include Acremonium, Alternaria, Aspergillus, Cladosporium, Fusarium, Mucor, Penicillium, Rhizopus, Stachybotrys, Trichoderma and Trichophyton. In the sense of the present invention, the term «mold» also comprises mildew, a closely related counterpart of mold, which is either species of fungus in the order Erysiphales, or fungus-like organisms in the family Peronosporaceae. Thirdly, the tarsonemid mites according to the invention do not cause significant plant damage, contrary to Polyphagotarsonemus latus and Phytonemus pallidus mites which are considered as sub-major pests at low elevations in summer months. Actually, mites from the species P. latus and P. pallidus attack many fruit and vegetable crops in temperate and subtropical areas. Accordingly, the composition according to the invention, contrary to mite compositions comprising Polyphagotarsonemus latus and Phytonemus pallidus mites allow both mass rearing and pest control.

Advantageously, the composition according to the invention may further comprise a fungus. Better efficiency results were observed when fungi are added to the composition according to the invention. In fact, the prey mite population feeds on fungi. Thus, the active addition of fungi in the composition according to the invention makes it possible to improve the growth and therefore to accelerate the reproduction maturity of Tarsonemidae, which will lead to a faster increase of Tarsonemidae individuals of the prey mite population. In addition, it has been observed that the active addition of beneficial fungi stabilizes the rearing cultures and further suppresses the growth of unwanted fungal contaminations. Accordingly, the active addition of fungi in the composition according to the invention will indirectly enhance the rearing of predator mite population, their large-scale production and the distribution of the composition according to the invention at larger volumes but will also show better effects on pest control on crops. In a preferred embodiment, the fungus belongs to the Hypocreaceae, in particular, the fungi are selected from Gliocladium and Trichoderma.

“Tarsonemid” as used herein refers to a mite species of the family of Tarsonemidae, both terms are used interchangeably herein. The tarsonemidae are a family of mites, also called thread footed mites or white mites. In a particular embodiment, the tarsonemid mites belong to the subfamily of Tarsoneminae, more in particular to the tribe of Hemitarsonemini, Steneotarsonemini, Tarsonemini, or Pseudacarapini.

In a preferred embodiment, the tarsonemid mites are selected from the group consisting of the genera Tarsonemus (e.g. Tarsonemus granarius, Tarsonemus floricolus, Tarsonemus myceliophagus, Tarsonemus subcorticalis, Tarsonemus minimax, Tarsonemus fusarii), Fungitarsonemus (e.g. Fungitarsonemus peregrinus, Fungitarsonemus pulvirosus), Heterotarsonemus (e.g. Heterotarsonemus lindquisti, Heterotarsonemus coleopterorum), Daidalotarsonemus (e.g. Daidalotarsonemus vandevriei, Diadalotarsonemus tesselatus), Neotarsonemoides (e.g. Neotarsonemoides denigrates), and Pseudacarapis (e.g. Pseudacarapis indoapis). In a more preferred embodiment, the prey mites of the invention are selected from the genus Tarsonemus; more in particular from the group consisting of Tarsonemus granarius, Tarsonemus floricolus, Tarsonemus myceliophagus, Tarsonemus subcorticalis, Tarsonemus minimax, and Tarsonemus fusarii.

Preferably, the tarsonemid mites according to the invention do not cause significant plant damage. The skilled person is well aware how to select tarsonemid mites that do not cause significant plant damage. In addition, the absence of causing significant plant damage is easily determined, e.g. by incubating a plant with the tarsonemid of choice and determining if the plant is damaged after a few days. In a particular embodiment, the prey mites according to the invention include mites from the family of the Tarsonemidae, wherein said mites from the family of the Tarsonemidae are not selected from the group comprising Polyphagotarsonemus latus and Phytonemus pallidus. As will be understood from the context herein, the tarsonemid mites according to the invention preferably are fungus-feeding tarsonemid mites.

Advantageously, the composition according to the invention comprises a food source for the predatory and/or prey mite populations. In a particular embodiment, the composition of the invention comprises a food source for said prey mite population. As also described elsewhere, the food source for said prey mite population may comprise a fungus. In another embodiment, the food source for said prey mite population comprises non-photosynthetic plant material. In a further embodiment, the food source comprises wheat germ or pollen. As is known to the skilled person, a carrier can be selected that acts both as a carrier and as a food source, for example wheat germ.

In a particular embodiment, the composition of the invention further comprises a supplemental food source for the predatory mite population. A supplemental food source as used herein refers to a food source that is present in addition to the prey mite population. Supplemental food sources are sometimes used in predatory mite rearing as a food source that is more cost-effective than prey mites. It is often observed that several predatory mites can use supplemental foods for obtaining additional energy, but also require the presence of prey mites for survival, development and reproduction. A major drawback of the use of a supplemental food source is that it is prone to fungal contamination. By combining a supplemental food source with the prey mites of the invention, this drawback is suppressed. Examples of preferred supplemental foods are natural food sources (such as pollen or dead mites) and artificial foods (Wackers et al. (2005) Plant-provided food for carnivorous insects: a protective mutualism and its applications 356p; Morales-Ramos et al. (2014) Mass production of beneficial organisms 742p; Cohen (2015) Insect diets. Science and Technology, Second edition, 473p) Supplemental food sources for the rearing of predatory mites are well-known to the skilled person.

As mentioned herein before, the carrier can be any solid material which is suitable to provide a carrier surface to the individuals. In a preferred embodiment, the carrier is a mixture of solid carrier elements. Preferably, the carrier is a substantially homogeneous mixture of solid carrier elements. While the solid carrier elements may have a variety of sizes, a substantially homogeneous mixture refers to the variety of sizes being substantially homogeneously distributed amongst the carrier.

The average longest axis of the solid carrier elements is typically between 1.0 and 20.0 mm, more in particular between 2.0 and 12.0 mm, preferably between 3.0 and 9.0 mm. In another particular embodiment, the carrier is a mixture of solid carrier elements with a similar size. In a further embodiment, 90% of the solid carrier elements have a longest axis in the range of 0.1 times to 10.0 times the average longest axis of the mixture of solid carrier elements, in particular in the range of 0.2 and 5.0 times, more in particular in the range of 0.5 and 2.0 times. As will be understood from the disclosures herein, two or more different types of carrier elements may be used and mixed. In such case, the size distribution refers to the size distribution per type of carrier elements. Preferably the carrier provides a porous medium, which allows exchanges of metabolic gases and heat produced by the mite populations. Examples of suitable carriers are non-photosynthetic plant material, such as (wheat) bran, buckwheat husks, rice husks, saw dust, corn cob grits, etcetera, or inorganic material, such as vermiculite. Advantageously, said carrier comprises grains of a grass species or any part thereof, such as germ or bran. Preferably, said carrier does not comprise living, green plant material, such as leaves or stems of plants. In a particular embodiment, the carrier comprises non-photosynthetic plant material. Particularly preferred is a carrier selected from vermiculite, wheat bran, millet chaff, rice husks and buckwheat husks.

Preferably, said carrier comprises carrier elements with an average longest axis of between 1.0 and 20.0 mm, in particular between 3.0 and 9.0 mm. The carrier material may provide shelter for the prey mites and predatory mites, reducing stress conditions and cannibalism. During mass rearing, the carrier allows for the generation of a three-dimensional rearing matrix and may provide a food source for the prey mites. During storage and distribution for biological control, the carrier acts as a bulking agent and allows for a more homogeneous distribution of the predatory mites in the crops.

Advantageously, the number of individuals of the predatory mite population relative to the number of individuals of the prey mite population is from about 1:1 to 1:500, such as about 1:50 to 1:200, and preferably between 1:75 and 1:125.

High densities of prey mites and predatory mites can be reached thanks to the present invention. In a particular embodiment, the mite compositions of the present invention comprise at least 1.000 mites of the prey mite population per gram of the composition, in particular at least 2.000 prey mites, more in particular at least 3.000 prey mites. In a further embodiment, at least 5.000 prey mites, at least 10.000 prey mites, at least 20.000 prey mites, at least 30.000 prey mites. In a preferred embodiment, the mite composition comprises at least 50.000 mites of the prey mite population per gram of the composition.

In another particular embodiment, the mite compositions of the present invention comprise at least 20 mites of the predatory mite population per gram of the composition, in particular at least 30 predatory mites, more in particular at least 50 predatory mites. In a further embodiment, at least 100 predatory mites, at least 150 predatory mites, at least 200 predatory mites, at least 300 predatory mites. In a preferred embodiment, the mite composition comprises at least 500 mites of the predatory mite population per gram of the composition.

Mite numbers are mentioned herein refer to the total number of all mite development stages, thus including eggs, larvae, nymphs and adults, unless the context clearly dictates otherwise. As will be understood from the disclosures herein, the prey mites in the compositions of the invention are not required to be living prey mites.

In a particular embodiment, the mite compositions of the present invention comprise at least 1.000 non-egg mites of the prey mite population per gram of the composition, in particular at least 2.000 non-egg prey mites, more in particular at least 3.000 non-egg prey mites. In a further embodiment, at least 5.000 prey non-egg mites, at least 10.000 prey non-egg mites, at least 20.000 prey non-egg mites, at least 30.000 prey non-egg mites. In a preferred embodiment, the mite composition comprises at least 50.000 non-egg mites of the prey mite population per gram of the composition.

In another particular embodiment, the mite compositions of the present invention comprise at least 20 non-egg mites of the predatory mite population per gram of the composition, in particular at least 30 non-egg predatory mites, more in particular at least non-egg 50 predatory mites. In a further embodiment, at least 100 non-egg predatory mites, at least 150 non-egg predatory mites, at least 200 non-egg predatory mites, at least 300 non-egg predatory mites. In a preferred embodiment, the mite composition comprises at least 500 non-egg mites of the predatory mite population per gram of the composition.

In another particular embodiment, the mite compositions of the present invention comprise at least 20 adult mites of the predatory mite population per gram of the composition, in particular at least 30 adult predatory mites, more in particular at least 50 adult predatory mites. In a further embodiment, at least 100 adult predatory mites, at least 150 adult predatory mites, at least 200 adult predatory mites, at least 300 adult predatory mites. In a preferred embodiment, the mite composition comprises at least 500 adult mites of the predatory mite population per gram of the composition. In further embodiment, the mite compositions of the present invention comprise at least 20 living adult mites of the predatory mite population per gram of the composition, in particular at least 30 living adult predatory mites, more in particular at least 50 living adult predatory mites. In a further embodiment, at least 100 living adult predatory mites, at least 150 living adult predatory mites, at least 200 living adult predatory mites, at least 300 living adult predatory mites. In a preferred embodiment, the mite composition comprises at least 500 living adult mites of the predatory mite population per gram of the composition.

Evidently, the above ranges of prey mite and predatory mite numbers can be combined to arrive to particular and preferred embodiments of the invention. It thus follows that the present invention provides, for example, mite compositions comprising at least 1.000 mites of the prey mite population and at least 20 mites of the predatory mite population per gram of the composition. As another example, at least 5.000 mites of the prey mite population and at least 100 mites of the predatory mite population. As another example, at least 50.000 mites of the prey mite population and at least 500 mites of the predatory mite population.

In a preferred embodiment, said predatory mites are mesostigmatid or prostigmatid mite species. In a further embodiment, said predatory mites are selected from:
Mesostigmatid predatory mite species such as:
i) Phytoseiidae such as from:
a)—the subfamily of the Amblyseiinae, such as from the genus Amblyseius, e.g. Amblyseius andersoni, Amblyseius aerialis, Amblyseius swirskii, Amblyseius herbicolus or Amblyseius largoensis, from the genus Euseius e.g. Euseius finlandicus, Euseius hibisci, Euseius ovalis, Euseius victoriensis, Euseius stipulatus, Euseius scutalis, Euseius tularensis, Euseius addoensis, Euseius concordis, Euseius ho or Euseius citri, from the genus Neoseiulus e.g. Neoseiulus barkeri, Neoseiulus califomicus, Neoseiulus cucumeris, Neoseiulus longispinosus, Neoseiulus womersleyi, Neoseiulus idaeus, Neoseiulus anonymus, Neoseiulus paspalivorus, Neoseiulus reductus or Neoseiulus fallacis, from the genus Amblydromalus e.g. Amblydromalus limonicus from the genus Typhlodromalus e.g. Typhlodromalus aripo, Typhlodromalus laila or Typhlodromalus peregrinus from the genus Typhlodromips e.g. Typhlodromips montdorensis, from the genus Phytoseiulus, e.g. Phytoseiulus persimilis, Phytoseiulus macropilis, Phytoseiulus longipes, Phytoseiulus fragariae;
b) the subfamily of the Typhlodrominae, such as from the genus Galendromus e.g. Galendromus occidentalism from the genus Typhlodromus e.g. Typhlodromus pyri, Typhlodromus doreenae, Typhlodromus rhenanus or Typhlodromus athiasae;
c) the subfamily of the Phytoseiinae, such as from the genus Phytoseius e.g. Phytoseius macropilis, Phytososeius finitimus or Phytoseius plumifer.
ii) Ascidae such as from the genus Proctolaelaps, such as Proctolaelaps pygmaeus (Muller); from the genus Blattisocius e.g. Blattisocius tarsalis (Berlese), Blattisocius keegani (Fox); from the genus Lasioseius e.g. Lasioseius fimetorum Karg, Lasioseius floridensis Berlese, Lasioseius bispinosus Evans, Lasioseius dentatus Fox, Lasioseius scapulatus (Kenett), Lasioseius athiasae Nawar & Nasr; from the genus Arctoseius e.g. Arctoseius semiscissus (Berlese); from the genus Protogamasellus e.g. Protogamasellus dioscorus Manson;
iii) Laelapidae such as from the genus Stratiolaelaps e.g. Stratiolaelaps scimitus (Womersley) (also placed in the genus Hypoaspis); Gaeolaelaps e.g. Gaeolaelaps aculeifer (Canestrini) (also placed in the genus Hypoaspis); Androlaelaps e.g. Androlaelaps casalis (Berlese);
iv) Macrochelidae such as from the genus Macrocheles e.g. Macrocheles robustulus (Berlese), Macrocheles muscaedomesticae (Scopoli), Macrocheles matrius (Hull);
v) Parasitidae such as from the genus Pergamasus; e.g. Pergamasus quisquiliarum Canestrini; Parasitus e.g. Parasitusfimetorum (Berlese), Parasitus bituberosus Karg; Parasitellus e.g. Parasitellus fucorum (De Geer);
vi) Digamasellidae such as from the genus Digamasellus; e.g. Digamasellus quadrisetus or Digamasellus punctum; from the genus Dendrolaelaps; e.g. Dendrolaelaps neodisetus; and
-prostigmatid mite species such as from:
i) Tydeidae such as from the genus Homeopronematus e.g. Homeopronematus anconai (Baker); from the genus Tydeus e.g. Tydeus iambi (Baker), Tydeus caudatus (Duges); from the genus Pronematus e.g. Pronematus ubiquitus (McGregor);
ii) Cheyletidae such as from the genus Cheyletus e.g. Cheyletus eruditus (Schrank), Cheyletus malaccensis Oudemans;
iii) Cunaxidae such as from the genus Coleoscirus e.g. Coleoscirus simplex (Ewing), from the genus Cunaxa e.g. Cunaxa setirostris (Rermann);
iv) Erythraeidae such as from the genus Balaustium e.g. Balaustium putmani Smiley, Balaustium medicagoense Meyer & Ryke, Balaustium murorum (Hermann);
v) Stigmaeidae such as from the genus Agistemus e.g. Agistemus exsertus Gonzalez; such as from the genus Zetzellia e.g. Zetzellia mali (Ewing); and
vi) Tarsonemidae such as from the genus Acaronemus, e.g. Acaronemus destructor (Smiley and Landwehr); such as from the genus Dendroptus near suski Sharonov and Livshits; such as Lupotarsonemus floridanus Attiah.

In a preferred embodiment, the predatory mites belong to the Phytoseiidae. In a particular embodiment, the predatory mites are selected from Amblyseiinae, Transeius, Neoseiulus and Amblydromalus; more in particular the predatory mites are selected from the group consisting of Amblyseius swirskii, Transeius montdorensis, Neoseiulus californicus and Amblydromalus limonicus.

In another embodiment, the predatory mites are prostigmatid mites, in particular Tydeidae. In a further embodiment, the predatory mites are from the genus Homeopronematus or Pronematus, preferably Homeopronematus anconai or Pronematus ubiquitus.

The present invention also provides a commercial packaging for storing and distributing the composition of the invention. Therefore, the present invention further pertains to a container comprising a composition according to the invention. In particular, the container has an internal volume of between 0.2 l and 3 l, preferably between 0.5 l and 2 l. According to a preferred embodiment the container preferably comprising an exit for at least one motile life stage of the mite, more preferably an exit suitable for providing a sustained release of said at least one motile life stage. In a particular embodiment, the container (e.g. a bottle or a sachet) has at least one exit with a removable seal. In particular, seal as used herein refers to a closure that is glued or heat-sealed. Preferably, the container is adapted to attach it to a crop, for example by comprising a hook to hang it from a crop leaf or branch or by comprising a sticky surface to stick it to a surface of the crop.

The present invention further pertains to a method for rearing predatory mites, the method comprising
providing a composition of the invention, and
allowing individuals of the predatory mite population to feed on said prey mite population.

Preferably, the individuals of the predatory mite population are allowed to feed on said prey mite population while maintaining said mite composition at 5 to 35° C. and 30 to 100% relative humidity; in particular at 15 to 35° C. and 50 to 100% relative humidity.

As described herein before, in another embodiment, the method for rearing predatory mites further comprises adding a fungus. In a particular embodiment, the fungus is a freeze-dried fungus. For example, present invention provides a method for rearing predatory mites, the method comprising:
generating a mite composition by contacting a carrier with a prey mite population, a predatory mite population, and a culture of fungus, and
allowing individuals of the predatory mite population to feed on said prey mite population. In principle, the fungus can be added at any time during the rearing method and will then provide benefits, during rearing and/or during use of the resulting composition for biological control. In a particular embodiment, the fungus is added such that the fungus is present while the individuals of the predatory mite population feed on the prey mite population. In a preferred embodiment, the fungus is added before the predatory mite population is allowed to feed on said prey mite population. In a further embodiment, the rearing method of the invention comprises:
contacting a carrier with a prey mite population and a fungus,
rearing said prey mite population on said carrier in the presence of said fungus,
contacting said reared prey mite population with a predatory mite population, and
allowing individuals of the predatory mite population to feed on said prey mite population.

Beneficially, the carrier can be incubated with a fungus before the prey mite population is added. This allows for the growth of the fungus on the carrier and ensures a sufficient food source for the prey mite population.

Advantageously, when the reared prey mite population is contacted with the predatory mite population, the prey mite population can be retained on the carrier and in the presence of the fungus. There is thus no need to isolate the prey mite population from the carrier and fungus before contacting it with the predatory mite population. In addition, the presence of fungus during rearing of predatory mites provides the benefits as described herein. In particular, the fungus may act as a food source for the prey mite population, such that the prey mite population can develop further while it is preyed upon by the growing predatory mite population.

The present invention further pertains to a use of one or more species of mite from the family of the Tarsonemidae according to the invention to control fungal growth. Advantageously, fungal growth is controlled in a predatory mite rearing system.

The present invention further pertains to a method for controlling a pest in a crop, the method comprising providing to said crop a mite composition according to the invention. The mite composition of the invention may be distributed directly onto the crop. Alternatively, the mite composition of the invention is provided in the proximity of the crop. For example, the mite composition of the invention may be provided to a crop by placing a container holding the mite composition in the vicinity of the crop and allowing the predatory mites to exit from said container. This way, predatory mites spread themselves through the crops and control the crop pests. In a preferred embodiment, the pest is an arthropod pest.

These and other embodiments of the invention are indicated in the appended claims.

The invention will now be further described with reference to the following examples, which show non-limiting embodiments of different aspects of the invention.

### EXAMPLES

### Example 1: Oviposition Rate of Amblyseius swirskii Athias-Henriot on Different Prey Mites

The first example was conducted to evaluate the nutritional quality of a tarsonemid prey mite as compared to two standard used astigmatid prey mites for a phytoseiid predatory mite. More specifically, the oviposition rate of the predatory mite Amblyseius swirskii Athias-Henriot (Acari: Mesostigmata: Phytoseiidae) was tested on the tarsonemid prey mite Tarsonemus fusarii Cooreman (TF) (Acari: Prostigmata: Tarsonemidae) as compared to two astigmatid prey mites, namely Carpoglyphus lactis L. (CL) (Acari: Astigmata: Carpoglyphidae) and Thyreophagus entomophagus (Laboulbene) (TE) (Acari: Astigmata: Acaridae).

Prey mites were cultured on a medium containing bran, wheat germ and yeast in plastic containers (8×5.7 cm) with a ventilated lid. The containers were maintained at a temperature of 22±1° C. and a relative humidity of 85±5%.

A single gravid female A. swirskii was transferred from mass-rearing facilities onto a black PVC arena (2×4 cm) placed on a layer of wet cotton. The edges of the arena were covered with tissue paper to prevent mites from escaping. A black piece of plastic (1×1 cm) with cotton threads was added to provide shelter and oviposition substrates. Prey mites were provided ad libitum. The number of eggs deposited by each female was counted daily during 4 consecutive days. The oviposition rate of the first day was omitted from analysis to limit the effect of the diet prior to the experiment (Sabelis 1990). Twelve replications were used for each treatment. The sum of the amount of eggs laid during day 2, 3 and 4 were compared with GLM model with Poisson distribution. Contrasts among treatments were determined with general linear hypothesis testing (function glht of the package Ismeans in R, Lenth 2016). All analyses were performed using the statistical software R 3.10 (RDevelopment Core Team 2012).

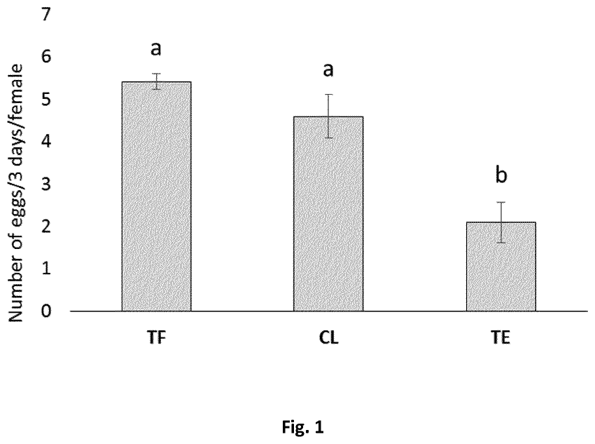

The results demonstrate that A. swirskii is able to reproduce on the tarsonemid prey mite T. fusarii. Egg-laying was influenced by the prey specie (GLM, χ2=20.3, d.f.=2, p<0.001) with that on C. lactis and significantly better than a diet consisting of T. entomophagus (FIG. 1). More eggs were laid when grown on the tarsonemid prey mite in comparison to the astigmatid prey mites, although this didn't reach statistical significance in relation to C. lactis. In conclusion, tarsonemid mites are a suitable prey mite population for the commercial rearing of predatory mites, such as A. swirskii.

### Example 2: Capture Success Rate of A. swirskii when Provided Different Developmental Stages of Prey Mites

Previous studies have reported that adult stages of astigmatid prey mites are more difficultly subdued and consumed than eggs and larvae of these mites. In the present example, the rate of a capture success rate was assessed for the eggs, larvae and adults of the tarsonemid prey mite T. fusarii, and the astigmatid prey mites C. lactis and T. entomophagus.

Predatory and prey mites were reared as described in example 1. Female A. swirskii were starved for 16 h prior to start of the experiment by transferring them to a black PVC plate without any food source. After 16 h, a single female A. swirskii was transferred to a black PVC arena as described in experiment 1. Prior to the introduction of the A. swirskii female, either 50 eggs, 50 larvae or 25 adults of the three prey mites were transferred to the experimental arena. After introduction of the predator, observations were done for 5 minutes or until a successful attack (i.e. killing and feeding on the prey). If a female did not succeed in killing prey within 5 minutes, this was recorded as unsuccessful. For each treatment, 15 replicates were set up. Data were compared among treatments with a GLM model with binomial distribution. Contrasts among treatments were assessed by stepwise model simplification through aggregation of nonsignificant factor levels (Crawley 2013). All statistical analyses were done using the computer software R version 3.1.0 (R Core Team 2014).

When tarsonemid T. fusarii eggs were provided, all females successfully fed on the eggs within 5 minutes. Surprisingly, less than 30% of the females were capable of killing a C. lactis egg and none of the females killed T. entomophagus eggs. For the adults, also a higher proportion of prey individuals were subdued when the tarsonemid prey mite T. fusarii was provided as compared to the other astigmatid prey mite species. For larvae, all females killed larvae within 5 minutes.

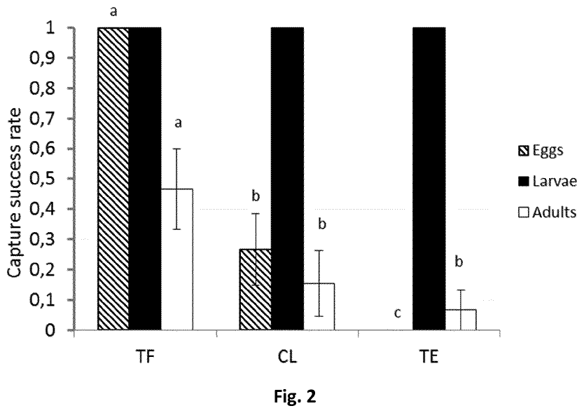

Capture rate was equal between all species when the prey were larvae (GLM, χ2=2×10-10, d.f.=2, p=0.05). However, the capture rate was influenced by species when eggs (GLM, χ2=1315.3, d.f.=2, p<0.05) and adults (GLM, χ2=39.2, d.f.=2, p<0.05) were offered. From the results presented in FIG. 2, it is clear that A. swirskii females are able to kill and consume much more of the eggs and adults of the tarsonemid prey mite T. fusarii as compared to the conventionally used astigmatid prey mite species C. lactis and T. entomophagus. Thus, for tarsonemid prey mites, all developmental stages are more easily subdued and killed, making it a more suitable prey.

### Example 3: Oviposition Rate of Homeopronematus anconai on Tarsonemus fusarii

This experiment was conducted to evaluate the nutritional quality of a tarsonemid prey mite for a prostigmatid predatory mite, namely Homeopronematus anconai (Baker).

Prey mites were cultured on a medium containing bran, wheat germ and yeast in plastic containers (8×5.7 cm) with a ventilated lid. The containers were maintained at a temperature of 22±1° C. and a relative humidity of 85±5%.

A single gravid female H. anconai was transferred from mass-rearing facilities onto a tomato leaf arena (2×4 cm) placed on a layer of wet cotton. The edges of the arena were covered with tissue paper to prevent mites from escaping. Prey mites were provided ad libitum.

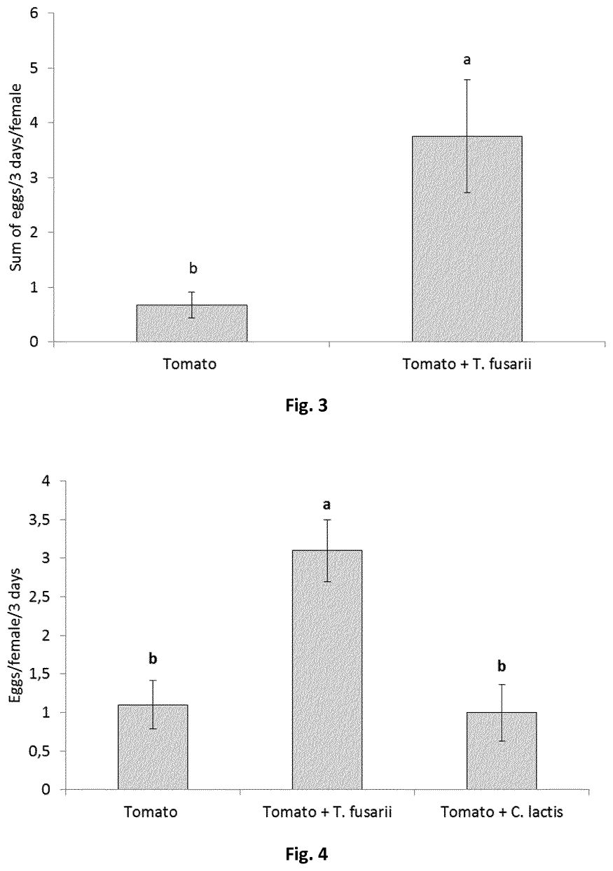

The number of eggs deposited by each female was counted daily during 4 consecutive days. The oviposition rate of the first day was omitted from analysis to limit the effect of the diet prior to the experiment (Sabelis 1990). Ten replications were used for each treatment. The sum of the amount of eggs laid during day 2, 3 and 4 were compared with GLM model with Poisson distribution using the statistical software R 3.10 (R Development Core Team 2012). Results are shown in FIG. 3. Providing T. fusarii on the tomato leaf arena resulted in a significantly higher number of eggs laid by H. anconai (GLM, χ2=20.418, d.f.=1, p<0.001), supporting that tarsonemids can be used as prey mites for rearing prostigmatid predatory mites.

### Example 4: Oviposition Rate of Pronematus ubiquitus on Tarsonemus fusarii and Carpoglyphus lactis

This experiment was conducted to evaluate the nutritional quality of a tarsonemid prey mite for another prostigmatid predatory mite, namely Pronematus ubiquitus (Baker).

Prey mites were cultured on a medium containing bran, wheat germ and yeast in plastic containers (8×5.7 cm) with a ventilated lid. The containers were maintained at a temperature of 22±1° C. and a relative humidity of 85±5%.

A single gravid female P. ubiquitus was transferred from mass-rearing facilities onto a tomato leaf arena (2×4 cm) placed on a layer of wet cotton. The edges of the arena were covered with tissue paper to prevent mites from escaping. Prey mites were provided ad libitum.

The number of eggs deposited by each female was counted daily during 4 consecutive days. The oviposition rate of the first day was omitted from analysis to limit the effect of the diet prior to the experiment (Sabelis 1990). Ten replications were used for each treatment. The sum of the amount of eggs laid during day 2, 3 and 4 were compared with GLM model with a quasipoisson distribution to correct for overdispersion using the statistical software R 3.10 (R Development Core Team 2012). Results are shown in FIG. 4.

A significantly higher oviposition rate of P. ubiquitus females was observed when fed with T. fusarii as compared to C. lactis and a control treatment without food (GLM, χ2=15.039, d.f.=2, p=0.002).

Tarsonemid prey mites thus have potential to be used in the rearing of prostigmatid predatory mites, such as P. ubiquitus, whereas traditionally used feed mites, such as C. lactis, do not.

### Example 5: Comparison of the Oviposition Rate of Amblyseius swirskii when Fed Tarsonemus confusus and Tarsonemus fusarii

As shown in examples 1 and 2, A. swirskii can be reared successfully on Tarsonemus fusarii. In the current example, the oviposition rate of Amblyseius swirskii on Tarsonemus confusus was tested in comparison to Tarsonemus fusarii.

Both tarsonemid mites were cultured on a medium containing bran, wheat germ and yeast in plastic containers (8×5.7 cm) with a ventilated lid. The containers were maintained at a temperature of 22±1° C. and a relative humidity of 85±5%.

A single gravid female A. swirskii was transferred from mass-rearing facilities onto a black PVC arena (2×4 cm) placed on a layer of wet cotton. The edges of the arena were covered with tissue paper to prevent mites from escaping. A black piece of plastic (1×1 cm) with cotton threads was added to provide shelter and oviposition substrates. Prey mites were provided ad libitum. The number of eggs deposited by each female was counted daily during 3 consecutive days. Per treatment, 12 replicates were set up. The oviposition rate of the first day was omitted from analysis to limit the effect of the diet prior to the experiment (Sabelis 1990). The sum of the amount of eggs laid during day 2 and 3 was compared using GLM analysis (with quasipoisson distribution). Contrasts among treatments were determined with general linear hypothesis testing (function glht of the package Ismeans in R, Lenth 2016). Analysis was performed using the statistical software R 3.10 (R Development Core Team 2012).

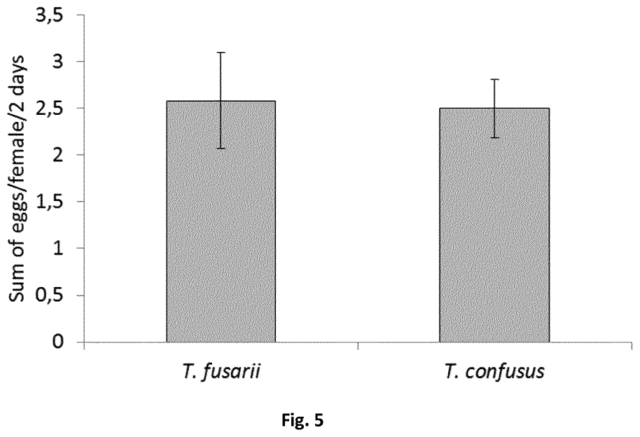

The results of the experiment are shown in FIG. 5. A similar number of eggs was laid by A. swirskii when offered either T. fusarii or T. confusus (GLM; χ²=0.0164, df=1, p=0.898). This result confirms that other tarsonemid species from the genus Tarsonemus, such as T. confusus, can be used as a food source for mass-production of predatory mites.

### Example 6: Oviposition Rate of Amblyseius swirskii on Fungitarsonemus sp.

This experiment was conducted to evaluate the nutritional quality of another tarsonemid prey mite, namely a Fungitarsonemus sp.

Fungitarsonemus sp. mobiles and eggs were collected from ivy leaves (Hedera helix L.). Prey mites were transferred to ivy leaf discs (2×4 cm) placed upside down on a layer of wet cotton. The edges of the leaf disc were covered with moist tissue paper to prevent the mite from escaping. A single gravid female A. swirskii, starved for 24 h prior to the experiment, was transferred to the leaf disc. Fresh Fungitarsonemus mobiles and eggs were transferred to the experimental leaf arena daily, in order to provide prey mites ad libitum. A black piece of plastic (1×1 cm) with cotton threads was added to provide shelter and oviposition substrates. The number of eggs deposited by the females was counted daily for two consecutive days. The leaf discs were kept in a climatic chamber set at a temperature of 22±1° C. and a relative humidity of 85±5%. Per treatment, 6 replicates were set up. The sum of the eggs laid during the two days was compared using GLM analysis (with poison distribution). Contrasts among treatments were determined with general linear hypothesis testing (function glht of the package Ismeans in R, Lenth 2016). Analysis was performed using the statistical software R 3.10 (R Development Core Team 2012).

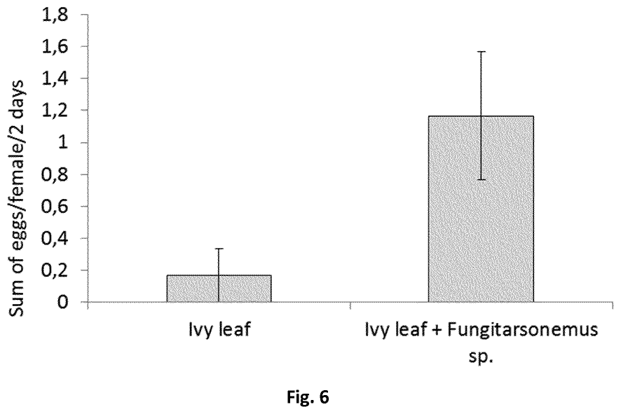

The results are shown in FIG. 6. A significant higher amount of eggs was counted when the Fungitarsonemus sp. was offered on the ivy leaf discs (GLM, χ²=5.062, df=1, p=0.0245). This confirms that tarsonemids outside of the Tarsonemus genus can be used for predatory mite rearing as well.

1. A mite composition comprising:
a predatory mite population,
a prey mite population, and
a carrier for individuals of said populations,
wherein said prey mite population comprises mites from the family of the Tarsonemidae,
provided that said mite composition does not comprise a living mite species selected from the group consisting of Polyphagotarsonemus latus and Phytonemus pallidus.

2. The composition according to claim 1, wherein said prey mite population comprises at least one mite species selected from the following genera:
the genus Tarsonemus, e.g. Tarsonemus granarius, Tarsonemus floricolus, Tarsonemus myceliophagus, Tarsonemus subcorticalis, Tarsonemus minimax; Tarsonemus fusarii;
the genus Fungitarsonemus, e.g. Fungitarsonemus peregrinus, Fungitarsonemus pulvirosus
the genus Heterotarsonemus, e.g. Heterotarsonemus lindquisti, Heterotarsonemus coleopterorum;
the genus Daidalotarsonemus, e.g. Daidalotarsonemus vandevriei, Diadalotarsonemus tesselatus;
the genus Neotarsonemoides, e.g. Neotarsonemoides denigratus
the genus Pseudacarapis, e.g. Pseudacarapis indoapis;

3. The composition according to claim 1, further comprising a food source for said mite populations.

4. The composition according to claim 1, wherein the food source for said prey mite population contains non-photosynthetic plant material, such as wheat germ or pollen.

5. The composition according to claim 1, wherein said carrier comprises grains of a grass species or any part thereof, such as germ or bran.

6. The composition according to claim 1, wherein said carrier comprises carrier elements with an average longest axis of between 1.0 and 20.0 mm.

7. The composition according to claim 1, wherein the number of individuals of the predatory mite species relative to the number of individuals of the prey mite is from about 1:1 to about 1:500; in particular from about 1:50 to about 1:200; more in particular between 1:75 and 1:125.

8. The composition according to claim 1, wherein said predatory mite species are mesostigmatid or prostigmatid mite species, in particular Phytoseiidae.

9. The composition according to claim 1, further comprising a fungus from the Hypocreacea.

10. A container comprising the composition of according to claim 1.

11. A method for rearing predatory mites, the method comprising
providing the composition of according to claim 1, and
allowing individuals of the predatory mite population to feed on said prey mite population.

12. The method of claim 11, further comprising maintaining said mite composition at 5 to 35° C. and 30 to 100% relative humidity.

13. A method comprising using one or more species of mites from the family of the Tarsonemidae selected genera from claim 1 to control fungal growth.

14. The method of claim 13, wherein fungal growth is controlled in a predatory mite rearing system.

15. A method for controlling a pest in a crop, the method comprising providing to said crop a mite composition according to claim 1.
